(19) United States
(12) Patent Application Publication
(10) Pub. No.: US 2025/0216332 A1
(43) Pub. Date: Jul. 3, 2025

(54) REAL-TIME RAMAN SPECTROSCOPIC MONITORING OF TEQUILA PRODUCTION

(71) Applicant: Endress+Hauser Optical Analysis, Inc., Ann Arbor, MI (US)

(72) Inventor: Sean J. Gilliam, Plymouth, MI (US)

(21) Appl. No.: 18/999,066

(22) Filed: Dec. 23, 2024

Related U.S. Application Data
(60) Provisional application No. 63/616,433, filed on Dec. 29, 2023.

(51) Int. Cl. G01N 21/65; G01N 33/14

(52) U.S. Cl. CPC G01N 21/65; G01N 33/146

(57) ABSTRACT

A method of characterizing and monitoring a fermentation process includes acquiring online Raman spectra of a hydrolysis process within a vessel at different times during the pressing process to generate a training data set; acquiring physical samples from pressing process near in time to the acquired Raman spectra; performing offline measurements of the target analyte properties and/or compositions using an assay measurement technique; generating a correlative model of the target analyte such that spectral changes in the training data set correlate with the offline measurements of the target analyte properties and/or compositions; acquiring online Raman spectra of a subsequent run of the pressing process within the vessel at different times during the run to generate a process data set; and applying the correlative model to the process data set to qualitatively and/or quantitatively predict a value of a property and/or composition of the target analyte.

### CROSS-REFERENCE TO RELATED APPLICATION

The present application is related to and claims the priority benefit of U.S. Provisional Application No. 63/616,433, filed Dec. 29, 2023, the entire contents of which are incorporated herein by reference.

### TECHNICAL FIELD

The present disclosure generally relates to Raman spectroscopy and, in particular, to a method applying Raman spectroscopy to tequila production processes in a large-scale production setting.

### BACKGROUND

Raman spectroscopy is a technique used to measure the wavelength and intensity of inelastically scattered radiation (e.g., light) from a sample, thereby revealing chemical and structural composition of the sample. Raman spectroscopy is based on the principle that monochromatic excitation light will be reflected, absorbed, or scattered as a function of a particular molecule (e.g., proteins, peptides, carbohydrates, acids, alcohols, etc.) that receives the incident radiation. Most of the energy is scattered at the same wavelength of the excitation light, referred to as elastic or Rayleigh scattering. A much, much smaller amount (e.g., ˜0.001%) is scattered at different wavelengths, called inelastic or Raman scattering, the wavelengths of which are dependent on, not just the molecular composition of the region sampled, but also the environment in which the molecule is analyzed. In Raman spectroscopic analysis (interchangeably, Raman analysis and Raman spectroscopy), the wavelength shifts of the inelastically, Raman scattered light are captured in Raman spectra comprising the Raman scattered light (i.e., the Raman signal), which are analyzed to determine sample properties, including both chemical and physical properties.

Generally, practical implementations of Raman analysis include data processing and modeling of the Raman spectra to separate and identify a relatively weak Raman signal of a target analyte, and then building a model that correlates orthogonal information to changes to the Raman signal to produce useful results upon which actions may be taken. Conventional Raman spectroscopy is not a “plug and play” technology unlike other conventional at-line and/or online analytical assays that combine a physical separation technique prior to a primary sensor. The technology relies on utilizing statistical algorithms to mathematically separate the individual constituent signals from the assortment of signals that are collected, which enables measuring multiple constituents simultaneously in complex environments and without the use of a physical separation technique.

A Raman probe can provide a molecular fingerprint or “Raman signature” relating to the vibrational spectroscopic information for all the molecular components within the sample. Raw spectroscopic data must be preprocessed to both enhance the signal of analytes and reduce external noise from other species present in the sample and, thus, the raw data. The preprocessed data is then modeled using statistical algorithms, such as univariate and/or multivariate analysis approaches (sometimes referred to as chemometric modeling), including but not limited to partial least squares (PLS), support vector regression (SVR), indirect hard modeling (IHM), locally weighted regression (LWR), principal component analysis (PCA), machine learning (ML) and any of its variants like deep learning (DL), artificial neural networks (ANN) and the like, to extract the maximum amount of relevant correlative information from the Raman spectral data. Using chemometric modeling, Raman analysis can provide multiple markers of the metabolic processes, which in turn, can be correlated and tracked in real-time both to offline measurements using known laboratory techniques (e.g., mass spectrometry, gas chromatography-mass spectrometry (GC-MS), liquid chromatography-mass spectrometry (LC-MS), high-performance liquid chromatography (HPLC), enzymatic methods, and the like) and to non-specific online methods (e.g., a wide variety of absorption-based techniques such as UV-Vis and fluorescence spectroscopy).

Tequila is an alcoholic beverage obtained from the distillation of fermented juice of the mature stems (called piñas) of Agave tequilana Weber var. azul, which are typically harvested once the total sugars within the stem meet a desired concentration. In general, there are three major stages in the tequila production process, namely agave juice (e.g., aguamiel) extraction (e.g., harvesting, milling, shredding, cooking and extracting, e.g., via hydrolysis), fermentation (e.g., agave must) and distillation (see FIG. 1), in addition to various processes associated with these three stages. In the first stage, the agave juice containing fermentable sugars is first obtained via hydrolysis using ovens or autoclaves or through a hot water diffusion process. In the former, agave stems are cooked in ovens or autoclaves at high temperatures (95-120° C.) for a long time (usually 8-12 hours) in the case of an oven, or 2-3 hours in the case of an autoclave. Once cooked, the water-soluble carbohydrates are extracted by simultaneous shredding and pressure washing followed by pressing. In the latter, raw agave juice is obtained from previously shredded raw agave stems using hot water (around 80° C.) through the use of equipment called a diffuser. Afterward, the carbohydrates contained in the raw agave juice are hydrolyzed for 4-6 hours under acidic conditions (pH 1.8-3) at high temperatures (80-85° C.). Considering that the main carbohydrate contained in agave is inulin, which is a polymer of fructose and glucose and is not susceptible to being fermented by yeasts, hydrolysis is needed to obtain simple, fermentable sugars (e.g., mainly fructose).

In the general second stage, the agave juice is subjected to an alcoholic fermentation process, wherein agave sugars are transformed to ethanol, carbon dioxide, and other compounds (e.g., aldehydes, esters, furans, and ketones) by the action of different microorganisms, particularly yeasts. In the general third stage, the fermented must may be subjected to a two-step distillation process to obtain tequila, which may be blended with demineralized water to yield the desired alcohol concentration (e.g., in percentage terms, as alcohol by volume, or proof), conventionally between 35 and 55% ABV (70-110 proof). The distillation stage may include filtration operations to clarify the distilled product, thereby removing unwanted compounds. Further, certain types of tequila may be aged in wooden containers, yielding a smoky finish and desired color. As described, each of the three major stages may include other process operations, each of which may be monitored using process control techniques.

Conventionally, aside from macro-scale process parameters such as pH, temperature, density, dissolved oxygen and dissolved carbon dioxide, the analytes involved in tequila production processes are infrequently measured because testing must be done offline. For example, pH, which may be measured online, is typically calibrated for drift via an offline assay. Due to collection time, personnel and cost, offline analytics continuous acquisition is impractical. These limitations deprive a distiller of the ability to observe, measure and correct real-time process deviations, which could lead to product quality issues and decreased yields. Aside from macro-scale process parameters, most other process parameters must be measured offline, or in certain cases, atline, using such techniques as GC-MS, LC-MS, HPLC, spectrophotometry, etc., as non-limiting examples.

Thus, there is a need for improvements in the area of process control of tequila-making processes.

### SUMMARY

One aspect of the present disclosure discloses a method of characterizing and monitoring one or more operations of a tequila production process, including hydrolyzation, fermentation and distillation.

In embodiments of the present disclosure, the method further comprises iteratively refining the correlative model using modeling statistics such that correlation of the training data set to the offline measurements is increased. In a further embodiment, the method comprises generating a signal, alarm or report when the value of the target analyte deviates from a desired range based on a threshold limit. In embodiments of the present disclosure, the target analyte properties and/or concentrations include at least one of: reducing sugars (e.g., fructose concentration, glucose concentration), degrees Brix, non-reducing sugars (e.g., inulin and sucrose concentrations), pH value, nitrogen content, volatile acidity (e.g., acetic acid, formic acid, butyric acid), non-volatile acids (e.g., gluconic acid and lactic acid concentrations), polyols (e.g., glycerol, etc.), volatile alcohols (e.g., ethanol, methanol and 1-propanol), aldehydes and esters (e.g., furfural, acetaldehyde, ethyl acetate, etc.), and volatile fusel alcohols (e.g., isobutanol, 2-methyl-1-butanol, and 3-methyl-1-butanol).

In another aspect of the present disclosure, a computer program product comprises a non-transitory machine-readable storage medium encoding instructions that, when executed by one or more programmable processors, cause the one or more programmable processors to perform operations comprising: acquiring online Raman spectra within a vessel at different times during the pressing process to generate a training data set; applying spectral preprocessing to the training data set such that non-correlative and covariant changes due to non-relevant species and/or properties are minimized and correlative changes due to a target analyte is amplified; generating a corelative model of the target analyte such that spectral changes of the target analyte properties and/or compositions in the training data set corelate with offline assay measurements of physical samples taken from the pressing process near in time to the acquired Raman spectra; acquiring a subsequent online Raman spectrum during a subsequent run of the pressing process within the vessel; and applying the correlative model to the subsequent online Raman spectrum to qualitatively and/or quantitatively predict a value of a property and/or composition of the target analyte. In certain embodiments, the target analyte includes at least one of inulin concentration, fructose concentration, glucose concentration, sucrose concentration, degrees Brix value, pH value, acetic acid concentration, gluconic acid concentration, lactic acid concentration, glycerol concentration, ethanol, methanol, furfural, acetaldehyde, ethyl acetate, 1-propanol, isobutanol, 2-methyl-1-butanol, and 3-methyl-1-butanol. In at least one embodiment, the operations further comprise generating a signal, alarm or report when the value of the target analyte deviates from a desired range based on threshold limits.

### DESCRIPTION OF THE DRAWINGS

The described embodiments and other features, advantages and disclosures contained herein, and the manner of attaining them, will become apparent and the present disclosure will be better understood by reference to the following description of various embodiments of the present disclosure taken in junction with the accompanying drawings, wherein:

FIG. 1 illustrates a conventional tequila production process;

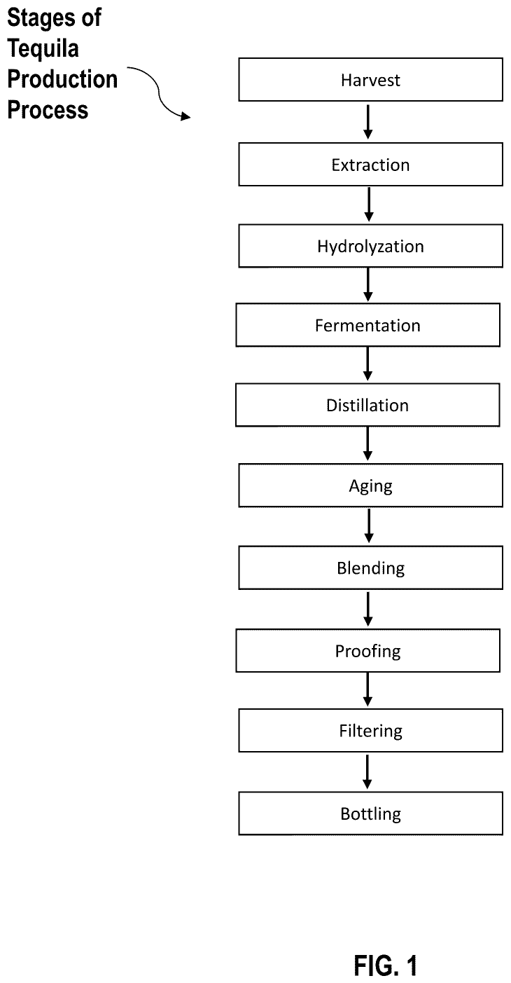

FIG. 2 shows a schematic representation of an embodiment of a Raman analysis system according to the present disclosure;

FIG. 3 shows an embodiment of a method of monitoring a hydrolyzation process for tequila production according to the present disclosure;

FIG. 4 shows a plot of raw Raman spectral data of multiple hydrolysis batches according to the present disclosure with absolute intensity in arbitrary units (a.u.) versus Raman shift (cm−1);

FIG. 5 shows a plot of preprocessed Raman spectral data for multiple hydrolysis batches according to the present disclosure with relative intensity in arbitrary units (a.u.) versus Raman shift (cm−1);

FIG. 6 shows a plot of a linear regression for inulin concentration prediction using an inulin correlative model according to the present disclosure with measured inulin concentration (% v/v) versus predicted inulin concentration (% v/v);

FIG. 7 shows a plot of a linear regression for fructose concentration prediction using a fructose correlative model according to the present disclosure with measured fructose concentration (% v/v) versus predicted fructose concentration (% v/v);

FIG. 8 shows a plot of a linear regression for glucose concentration prediction using a glucose correlative model according to the present disclosure with measured glucose concentration (% v/v) versus predicted glucose concentration (% v/v);

FIG. 9 shows a plot of a linear regression for sucrose concentration prediction using a sucrose correlative model according to the present disclosure with measured sucrose concentration (% v/v) versus predicted sucrose concentration (% v/v);

FIG. 10 shows a real-time prediction plot of inulin concentration (% v/v) during multiple hydrolysis batches over time (t) using the inulin correlative model in FIG. 6;

FIG. 11 shows a real-time prediction plot of fructose concentration (% v/v) during multiple hydrolysis batches over time (t) using the fructose correlative model in FIG. 7;

FIG. 12 shows a real-time prediction plot of glucose concentration (% v/v) during multiple hydrolysis batches over time (t) using the glucose correlative model in FIG. 8;

FIG. 13 shows a real-time prediction plot of sucrose concentration (% v/v) during multiple hydrolysis batches over time (t) using the sucrose correlative model in FIG. 9;

FIG. 14 shows an embodiment of a method of monitoring a fermentation process for tequila production according to the present disclosure; and

FIG. 15 shows an embodiment of a method of monitoring a distillation process for tequila production according to the present disclosure.

### DETAILED DESCRIPTION

The present disclosure includes the use of dispersive Raman spectroscopic analysis to perform real-time, non-destructive compositional analysis for tequila production, hydrolyzation, fermentation and distillation in particular. Creating a tequila's uniqueness and ensuring the final product matches a desired taste profile requires monitoring inputs to the process and during each stage of process. Both agricultural and process-related variations in agave juice attributes, particularly after hydrolyzation, and other inputs such as bacteria and yeast (e.g., natural and/or artificial) can significantly impact a tequila's quality. Having real-time, online measurements for process and quality control purposes improves yields and expedites the detection of quality issues during the fermentation process, which further reduces the cost of production.

Distilled agave spirits are produced and sold under several protected appellations of origin, for example, Tequila, Chipas, Mezcal, Sotol, and Bacanora, including categories, such as mixtos and 100% agave, bianco, reposado, añejo and extra añejo. In the present disclosure, the term “tequila” is used to refer to these and all types of distilled agave beverages.

The terms “inline”, “online”, “atline” and “offline” refer to different approaches to sampling and analysis in industrial and commercial processes. Generally inline refers to sampling and analysis in real time directly in the production process. Inline devices are integrated directly into a process stream and may perform continuous measurements without interrupting the flow of the material to be analyzed. Inline devices and measurements may also be referring to as in situ.

Online refers to sampling and analysis performed while a process is running, but samples are usually taken from a process stream and transported to an analyzer located in close proximity, for example, via a sampling interface, e.g., a flow cell. For example, online devices may be integrated into a sampling bypass line of the process and may perform measurements without interrupting the flow of the material. As used in the present disclosure, inline and online are used interchangeably to refer to sampling and analysis performed without interrupting a process.

Atline sampling and analysis refers to situations in which samples are taken from a process and manually transported to an analyzer not located directly at the process but is still nearby (e.g., in a quality control laboratory within a manufacturing facility). Offline sampling and analysis refers to taking samples from the process and transporting the samples a remote laboratory to be analyzed. Generally, offline sampling and analysis requires extra some time, and results are therefore not immediately available to influence the process. Nonetheless, offline analyses can often provide greater detail and accuracy because the analyses may be performed in a controlled laboratory environment using multiple measuring devices.

Raman spectroscopy has many unique advantages that make it ideally suited to process control applications. Numerous organic and inorganic materials may be non-destructively and non-intrusively investigated in solid, liquid and vapor phases, without sample preparation, often in real time. Excitation radiation and returned scattered radiation can be transmitted through wavelength-transmissive windows directly into and out of process vessels and via optical fibers over long distances for remote analysis. However, because the Raman effect is relatively weak, its applicability to certain in-line process control applications requiring fast acquisition times may be somewhat hindered. Reliable detection and quantification using Raman analysis requires sensitive and highly optimized instrumentation. Issues with excitation induced fluorescence radiation generated within a sample by the matrix or the sample itself, must also be considered.

Due to the weakness of the Raman effect, narrow-beam, high-intensity light sources (e.g., lasers) are one of the requirements needed to produce quality Raman spectra from a sample. To large extent, the resolution of the Raman spectrum relies on the bandwidth of the laser source used. Continuous wave laser sources are preferred, as pulsed lasers are more costly, more complex as well as utilize higher peak powers for sufficient signal-to-noise ratios, which have the propensity to damage samples. The choice of light source wavelength depends on the requirements of a given application. Lower visible wavelengths and upper UV (e.g., approximately 300 nm to 500 nm) may induce strong autofluorescence in organic materials, which can mask the relatively weak Raman signal. As such, longer visible or near-IR sources (e.g., 633-1064 nm) may be better suited for organic targets. However, the Raman signal intensity is inversely proportional (to the fourth power) to excitation wavelength. Accordingly, longer wavelengths can require longer acquisition times, which can be problematic in certain process control applications.

### Hydrolyzation

The present disclosure includes methods for using Raman analysis to model and determine the presence and concentration of multiple analytes within the agave juice hydrolyzation process while the hydrolyzation process is in progress (i.e., inline and/or online). Using the disclosed methods, the analyte determinations may be performed in real time in a non-destructive and immersive manner, e.g., within a process vessel (e.g., a cooker, oven, autoclave, diffuser, etc.), without any sample preparation for Raman analysis. The present disclosure enables a tequila producer (often generally called a tequila distiller) to monitor and adjust process-specific parameters of a hydrolyzation process to fine-tune agave aguamiel characteristics and yields in real-time, particularly with respect to non-reducing and reducing sugar concentrations as well as pH value.

The raw materials to produce distilled beverages contain carbohydrates, which need the process of hydrolysis to obtain fermentable (reducing) sugars (e.g., mostly fructose). These carbohydrates may be a whole set of different compounds, from which problems can arise when hydrolyzing them due to their different conditions and properties (e.g., short, long, and branched chains). Single chain carbohydrate sugars can caramelize during complex chain hydrolysis, and during this reaction, secondary compounds such as furfural are generated. In addition, due to normal variability in the incoming agave feed, a hydrolysis process end point is difficult to optimize without real-time process feedback. Thus, the hydrolysis stage of the process is considered complicated, and good control of this operation is needed to avoid excessive production of undesirable compounds. Hydrolysis and hydrolyzation (i.e., the process of hydrolysis) are used interchangeably in the present disclosure.

Inulins are a group of naturally occurring polysaccharides produced by agave plants and which belong to a class of dietary fibers known as fructans. Inulin, which is considered a non-reducing sugar, is used by agave plants as a means of storing energy, which can be converted to fermentable, reducing sugars, such as fructose and glucose, by hydrolyzation. Consequently, inulin concentration is a key input characteristic and process control parameter for producing agave aguamiel. Further, furfurals (e.g., hydroxymethyl furfural) are aldehyde by-products of hydrolysis that may be involved as a fermentation inhibitor and should be controlled.

The aroma and flavor of tequila are the result of many volatile compounds that may be contained in the raw agave material and that may vary between harvests and batches. The aromas and flavors are further increased and transformed during the cooking of the agave fructan-rich tissues during hydrolyzation, previous to performing the fermentation step. Determining and monitoring these many volatiles can be a key process and quality control. During the hydrolyzation step of tequila production, there are several key analytes that affect the quality of the current batch, and certain species are recognized as being important in the hydrolyzation of agave stems, for example, in defining the aroma and flavor of the end tequila product.

Excessive hydrolysis can generate problems with methanol, which is generated by demethylation of pectins present in agave fiber. However, poor hydrolysis means loss of sugars due to partial hydrolysis of carbohydrates. As is known, methanol can form bonds with ethanol, thereby producing “a new molecule,” and therefore, it can be difficult to separate methanol from ethanol using conventional methods despite the fact that they have different physical properties. Moreover, during hydrolysis, glycoproteins are formed (e.g., union of glucose and a protein), some of which are responsible for providing flavors and aromas characteristic of tequila but concurrently represent losses to the process.

### Fermentation

The present disclosure includes methods for using Raman analysis to model and determine the presence and concentration of multiple analytes within the agave base fermentation process while the fermentation process is in progress (e.g., online and inline). The agave fermentation process of the agave base is mostly an anaerobic process but may be aerobic at least at times. Using the disclosed methods, the analyte determinations may be performed in real time in a non-destructive and immersive way within the fermenter without any sample preparation for Raman analysis. The disclosed methods have proven effective in modeling and real-time prediction of multiple compositional characteristics during several fermentation validation batches. The present disclosure enables a tequila producer to monitor and adjust process-specific parameters of the fermentation process to fine-tune product quality and yields in real-time both throughout the tequila production process and in the end product. For example, process inputs such as the fermentable sugars, fermentation yeasts, and nutrients may be monitored and controlled. The present disclosure further enables reduced production costs in manufacturing, real-time fault monitoring for non-ideal batches, and improved critical quality attributes of the product produced. Aldehydes (along with ketones) are perceptible by-products of fermentation, where a lack of nutrients or any factor that reduces the viability or activity of the yeasts generates an increase in the concentration of aldehydes. Nonetheless, aldehydes provide much of the aromatic profile of distilled beverages generally. A correct proportion of aldehydes in the finished product may have a pleasant aroma; a very high amount of aldehydes can cause unpleasant rotting odors.

During the agave must fermentation step of tequila production, there are several key analytes that affect the quality of the current batch, and certain species are recognized as being important in the fermentation of agave must, for example, in defining the aroma and flavor of the end tequila product.

### Distillation

The present disclosure includes methods for using Raman analysis to model and determine the presence and concentration of multiple analytes within the tequila distillation process at one or more stages of the process while in progress (e.g., inline and online). Tequila is distilled twice at minimum per governmental regulations to separate the constituents of the fermented wort (also called “mosto”). For example, in a first cycle, which yields the “ordinary,” undesirable components are eliminated (e.g., via destruction or depletion), such as the remains of yeasts, nutrient salts, solids, some secondary alcohols such as methanol and a group of compounds known as higher alcohols. Such undesirable components are often known as vinasses. In a second cycle of distillation, the ordinary is subjected to rectification to obtain the most volatile compounds of the liquid mixture (i.e., enrichment), which are then condensed and converted to liquid form as higher alcohol content tequila.

Using the disclosed methods, the analyte determinations may be performed in real time in a non-destructive and immersive way within a distillation column without any sample preparation for Raman analysis. The present disclosure enables a tequila distiller to monitor and adjust process-specific parameters of the distillation processes to fine-tune tequila characteristics and yields in real-time.

During the distillation step of tequila production, there are several key analytes that affect the quality of the current batch, and certain species are recognized as being important in the distillation of agave must, for example, in defining the aroma and flavor of the end tequila product.

A final, distilled tequila may then be matured or aged for some period (e.g., in wood (e.g., charcoalized oak) containers). The temperature, humidity, initial alcoholic content of the tequila, and the time and the number of cycles of the wood containers influence the color, aroma and final flavor of the tequila. Changes during the maturation of tequila are, at least in part, caused by: (1) a decrease in the higher alcohols, which may be absorbed by the charcoal of the wood containers and smoking the final product; (2) extraction of compounds from the wood itself that provide the particular color and aroma (e.g., tannins); (3) reaction between some components of the distilled tequila giving rise to new ones; and (4) oxidation of the original components of tequila and those extracted of wood. The methods of the present disclosure may be employed to control and monitor the distilled tequila product during maturation.

Governmental regulatory agencies have set limits for certain compounds in distilled beverages, such as shown in Table 1, which are established, e.g., in Mexico, in standards NOM-142-SSA1/SCFI-2014.

TABLE 1
Regulatory Limits

| Analyte | Molecular Weight | Limit |
|---|---|---|
| Methanol | 32.04 | 3000 |
| Furfural | 96.08 | 40 |
| Acetaldehyde | 44.01 | 400 |

For the purposes of promoting an understanding of the principles of the present disclosure, reference will now be made to the embodiments and examples illustrated in the drawings, and specific language will be used to describe the same. It will nevertheless be understood that no limitation of the scope of this disclosure is thereby intended.

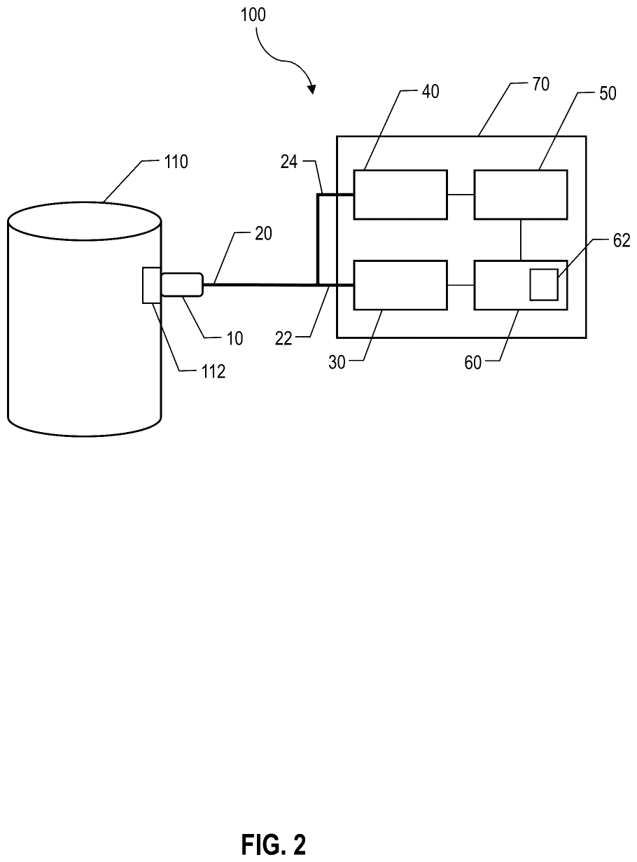

According to at least one embodiment of the present disclosure, a Raman system 100 for monitoring hydrolyzation, fermentation and/or distillation processes is shown in FIG. 2. The structure of the Raman system 100 will be described as applied to a fermentation process, including a process vessel 110, e.g., a container, tank, pipe, pipeline, process line, or flow cell, as non-limiting examples. Nonetheless, the Raman system 100 also may be employed for monitoring hydrolyzation, distillation, and/or the aging processes in different containers, reactors, pipelines and process vessels as described further herein.

The Raman system 100 may include a fiber-coupled Raman probe 10, which is configured and arranged to transmit excitation light into the fermentation process within a fermentation vessel 110 and to collect a Raman signal emitted from a sample volume within the fermentation vessel 110. The probe 10 may be coupled to an excitation light source 30 by a fiber optic cable 20 such that excitation light emitted by the light source 30 is incident upon a sample within the sample volume via a process connection 112. In at least one embodiment, the process connection 112 includes an opening and is configured to enable attachment of the probe 10 to the fermentation vessel 110 such that the probe 10 is in direct contact with the sample via the opening of the process connection 112. In a further embodiment, the process connection 112 may include a window that is transparent to the excitation light of the light source 30 and the Raman signal such that the probe 10 is in indirect contact with the sample via the window of the process connection 112. The process connection 112 may be any suitable means of arranging the probe 10 such that the excitation light is incident upon the sample and the resulting Raman signal is incident upon the probe 10. The probe 10 and fiber optic cable 20 are configured to enable the Raman system 100 to excite the sample volume and collect the resulting Raman signal in situ within the fermentation vessel 110 while the fermentation process is in progress, thereby generating real-time data from the fermentation process in progress in a production-scale environment.

The probe 10 may be any suitable, commercially available Raman probe, including those manufactured and sold by Endress+Hauser Optical Analysis, Inc. of Ann Arbor, MI (Applicant). The probe 10 may include a probe head in which various optical components are housed including, as non-limiting examples, sampling optics, gratings, filters, beam combiners and connectors configured to join the various optical components with the fiber optic cable 20. The fiber optic cable 20 may include a multi-mode fiber or a single-mode fiber, either of which is configured to provide an excitation path from the light source 30 to the probe 10, which conveys the excitation light to the sample, and a collection path from the probe 10 to a Raman instrument 70, which conveys scattered light containing the Raman signal from the sample to the Raman instrument 70. In at least one embodiment, the fiber optic cable 20 may include an excitation cable 22 for the excitation path and a separate collection cable 24 for the collection path in which the excitation and collection paths may be combined at or in the probe 10 using a combination of optical components including, for example, a grating and a beam combiner. For example, a beam combiner (e.g., a holographic beam combiner) in the probe 10 may be used to combine the excitation path onto a common optical axis with the collection path such that a common sampling optic in the probe 10 may be used for both paths such that a counter-propagating beam is formed within the probe 10.

In at least one embodiment, the Raman system 100 configured for process monitoring applications may include significant lengths (e.g., hundreds of meters) of the fiber optic cable 20 between the Raman instrument 70 and probe 10. Each optical fiber has its own Raman and elastic scattering signatures, which may be removed at the probe 10 before the excitation is emitted into the sample, for example, via the excitation cable 22. For example, a strong light source line due to Rayleigh scatter (i.e., the Rayleigh line) from the sample may be removed from the collected Raman signal at the probe 10 before entering the fiber optic cable 20 (e.g., the collection cable 24). Otherwise, the Rayleigh line may generate a competing fiber signature en route to the Raman instrument 70. In certain embodiments, a holographic grating and/or spatial filter may be inserted into the excitation path to remove the fiber signature. The Rayleigh line may be removed from the collected Raman signal by a notch filter (e.g., a holographic notch filter) in the collection path.

In certain embodiments, the Raman system 100 may be configured to monitor multiple sample locations in a process. In such an embodiment, multiple remote probes 10 may be coupled to a single Raman instrument 70 via multiple fiber optic cables 20. As shown in FIG. 2, the Raman instrument 70 may include the light source 30 and a spectrograph 40 configured to spectrally separate the Raman signal transmitted via the fiber optic cable 20 and to feed the spectrally separated Raman signal to a detector 50 of the Raman instrument 70 via optical components of the spectrograph 40. The Raman system 100 is a dispersive Raman system and includes a diffraction grating and corresponding optical components to disperse (e.g., spectrally separate) the Raman signal onto the detector 50. As a dispersive system, the Raman system 100 does not require moving components that can be affected by environmental conditions and other known disadvantages as such alternative systems as Fourier Transform Raman spectroscopy, for example.

The light source 30 may be a narrow-band, high-intensity light source including, but not limited to a laser, a laser diode, neon-tungsten lamp, mercury lamp or the like. The detector 50 may be an array detector (e.g., multi-channel) such as, but not limited to, a charge-coupled device (CCD) or a semiconductor array (e.g., a germanium or indium gallium arsenide (InGaAs) detector). In certain embodiments, the Raman instrument 70 may include a notch filter, an edge pass filter, a band pass filter and/or other optical components configured and arranged to remove the strong, elastically scattered (unshifted) Rayleigh component of the light transmitted to the spectrograph 40 that would otherwise hide (e.g., as noise) the relatively weak Raman signal. In embodiments employing multiple probes 10, the Raman instrument 70 may include a sequencer or splitter to multiplex the output of the light source 30.

The Raman instrument 70 may further include a controller 60 running software 62 configured to control the Raman system 100, including the light source 30, spectrograph 40 and detector 50, and to receive and analyze detector data from the detector 50 in communication therewith. The controller 60 may be configured for data acquisition and signal processing of the detector data to execute the methods of the present disclosure. The controller 60 may be configured to perform certain operations comprising a control structure to provide the functions described herein. In certain embodiments, the controller 60 forms a portion of a processing subsystem that includes one or more computing devices having memory, processing, and/or communication hardware. The controller 60 may be a single device housed within the Raman instrument 70 or a distributed device, and the functions of the controller 60 may be performed by hardware and/or software. The controller 60 can include one or more Arithmetic Logic Units (ALUs), Central Processing Units (CPUs), memories, limiters, conditioners, filters, format converters, or the like which are not shown to preserve clarity. In at least one embodiment, the controller 60 is programmable to execute algorithms and processes data in accordance with operating logic that is defined by programming instructions, such as software or firmware. Alternatively or additionally, operating logic for the controller 60 can be at least partially defined by hardwired logic or other hardware, for example, using an Application-Specific Integrated Circuit (ASIC) of any suitable type. The controller 60 can be exclusively dedicated to the functions described herein or may be further used in the regulation, control, and activation of one or more other subsystems or aspects of the Raman system 100.

The controller 60 may include one or more modules structured to functionally execute the operations of the controller 60. The description herein including modules emphasizes the structural independence of the aspects of the controller 60 and illustrates one grouping of operations and responsibilities of the controller 60. Other groupings that execute similar overall operations are understood to be within the scope of the present disclosure. Modules may be implemented in hardware and/or software on computer readable medium, and modules may be distributed across various hardware or software components.

The Raman system 100 is capable of both remotely delivering the excitation light to a particular process location and remotely collecting the Raman signal. With the capability of the Raman system 100 to detect chemical and physical information characteristic of a substance, when used to analyze any process, the chemical, physical and compositional information can be gathered in real-time, at any feasible point in the process and in a non-destructive manner.

In embodiments of the present disclosure, this real-time information feedback provides several advantages, including: (1) real-time feedback control and process optimization of multiple tequila production processes by monitoring actual chemical, physical, and compositional information; (2) real-time predictive determination of both the starting and end-point for individual properties; (3) end-of-batch property determination to forgo additional offline testing, thereby enabling real-time product release; and (4) the ability to compose and apply both specific and universal, non-varietal specific, scale-transferable models for prediction of each analyte within a future process, for example, a hydrolyzation, fermentation, and/or distillation process.

With the capability to collect spectral signatures of multiple physical and chemical properties in real time within the same spectrum, embodiments of the Raman system 100 extract quantitative compositional, chemical and physical information regarding various process parameters and several key analytes. If subsequent offline assay reference measurements are made, correlations may be established with respect to a composition and/or a particular property and how those relative changes relate to the acquired Raman spectrum, whether the spectrum was acquired in real time or post run.

Such correlations may be conducted in a univariate and/or multivariate space to relate online Raman spectral acquisitions to a discreet quantifiable compositional, chemical and physical properties acquired via traditional offline/at-line assay measurements. Spectral preprocessing, along with multivariate analysis, such as PLS, SVR, LWR, PCA, ML, DL, ANN and/or IHM, for example, provides information that may be plotted on a time scale to inform and alert an operator, in real time, of the quantifiable and/or the relative status of important constituents during and after each tequila production unit operation.

### Hydrolysis Process Implementation

According to an embodiment of the present disclosure, during one or more hydrolysis batches (e.g., individual instances of the hydrolysis process from start to finish), Raman spectra are acquired at specified intervals during each run from the process vessel 110 (e.g., an oven, autoclave, diffuser, etc.) using the Raman system 100. During and/or after the hydrolysis process, a number of samples are quantitatively measured via offline and/or atline assays. At the specific sampling times, the acquired Raman spectra of the samples are used to build one or more quantitative models that correlate each composition and/or property to changes of Raman spectral features (e.g., peaks (local maxima) and valleys (local minima)) occurring within a particular run or batch. In a possibly less correlative modality (relative to online dynamic measurements), samples may be extracted then analyzed by Raman analysis offline and then correlated to their respective offline assay values in at least on embodiment of the present disclosure. After the quantitative model is generated, the model may be applied in real time using an embodiment of the Raman system 100, including the probe 10 in communication with the hydrolysis process being characterized, monitored, and/or controlled.

The models of the present disclosure, as described further herein, are statistical, chemometric models, guided by first principles, of spectral features known to be associated with specific constituents (e.g., analytes, components, reactants, products, etc.) within certain frequency bands and under certain conditions. One skilled in the art of Raman spectroscopic analysis, having the benefit of the present disclosure, will recognize that the statistical modeling process enables the analysis of stages of a process, such as tequila production, without the need to directly, separately identify a molecular signature for each constituent in the process, which molecular signatures can themselves change as conditions (e.g., temperature, pressure, concentrations of other constituents, etc.) of the process change.

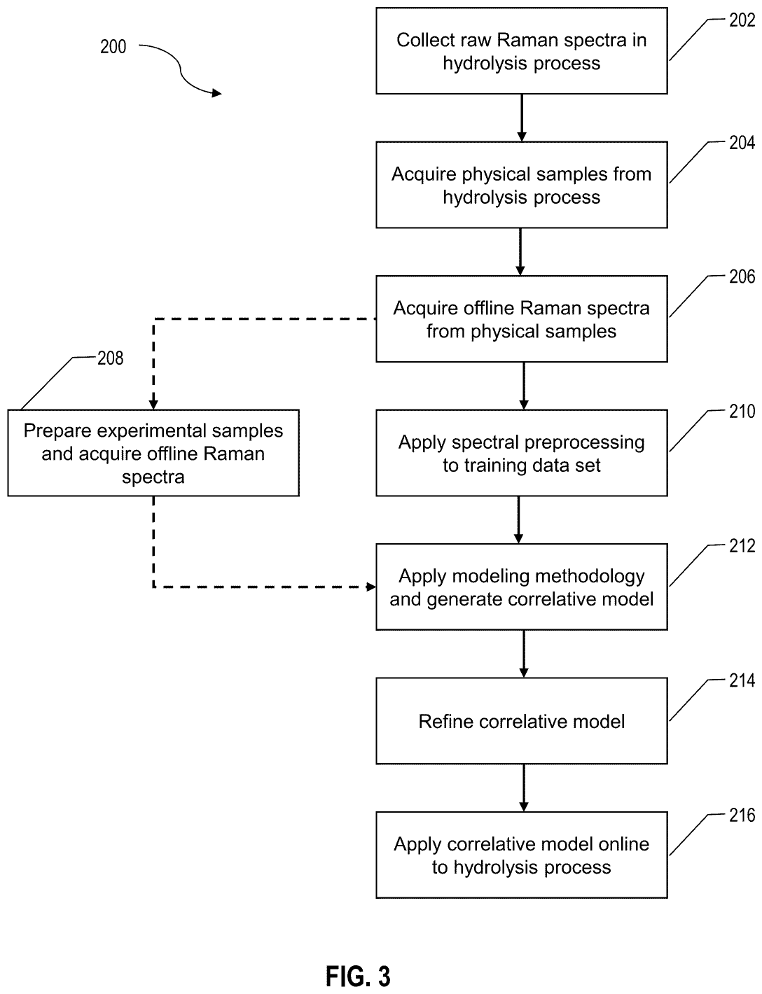

According to at least one embodiment of the present disclosure as shown in FIG. 3, a method 200 of characterization, monitoring and controlling a hydrolysis process includes generating a training data set, developing a chemometric, correlative model using the training data set, validating the model, and applying the model to future production hydrolysis process batch runs to characterize, monitor and control the production hydrolysis batch. The method 200 may include a step 202 of generating the training data set by spectrally collecting a statistically large set of online or inline process spectral measurements of raw Raman spectra at different points in time while the hydrolysis process is in progress (e.g., in the process 110 of FIG. 2) using the Raman system 100. The process measurements comprising the training data set include spectral variances of the constituents of the process during the hydrolysis process, including the analyte properties and compositions to be predict in future batches. Variance in the analytes enables the development of a robust model capable of accurately and precisely predicting future analyte properties and compositions within a range that will be measured in future batch runs. The online or inline process measurements may be time stamped to facilitate correlation to offline and/or atline measurements. The process measurements may be captured using a pre-determined amount of total acquisition time selected to generate a signal-to-noise ratio that is sufficient to produce the required measurement accuracy and precision.

Natural variance between hydrolysis batches lead to absolute differences in both Raman and autofluorescence signals acquired during a Raman exposure. For example, variance between agave varietals and growing conditions create variation in the concentrations of different precursor carbohydrates in the agave inputs to the hydrolysis process. The step 202 may require the Raman spectral exposure be dynamically adjusted for an optimized value during online sampling of the hydrolysis batch due to such natural variance. In addition, the precision of the Raman model is dependent on the quality of the Raman spectral data within the training date set. To produce data that is precise for a given set of requirements, the Raman signal may be maximized (to a point) whereas the noise of that measurement is reduced (to a point) as to provide an adequate signal-to-noise ratio (S/N ratio). A Raman absolute signal is linearly related to total acquisition time of the Raman signal, which is defined as the total amount of acquisition time in building one individual Raman spectrum from one or more exposures. A conventional rule of thumb instructs an operator to select an exposure time that is long enough to provide an adequate S/N ratio given the rate of dynamic change of the species (e.g., analytes) measured (sometimes called “temporal error”). To reduce the temporal error in predicted values from the Raman model, the selected total acquisition time should be optimized around the various rates of change of the measured analytes within the fermenter, knowing Raman analysis has an averaging effect of the predicted value over the time of the measurement. After the exposure and total acquisition time settings are determined given the desired precision and are optimized in view of the rate of change of the measured analytes, online process spectral measurements that capture the variance taken and analyzed to develop the model.

Depending upon the requirements of the Raman method, in at least one embodiment, a statistically large set of process samples may include a variety of independent samples of the hydrolysis process to enable one to predict a process value in which the variation in the modeled amounts and effects are less than 10% relative error to the “true” accepted value at that point in time. In other embodiments, the statistically large set of process samples may include enough independent samples of the process to enable one to conclude the variation in the modeled amounts and effects are at or under 5% relative error.

The method 200 may include a step 204 of acquiring physical samples during the hydrolysis process (e.g., from the process vessel 110) and performing offline and/or atline measurements of the analyte properties and/or compositions using an assay measurement device conventionally used to produce such values, including those prior art (e.g., non-Raman) devices and means described herein. As a non-limiting example related to the hydrolyzation stage, physical samples may be acquired from the hydrolysis process batch every few minutes as the process progresses (e.g., depending on which extraction process is selected). In at least one embodiment, the physical samples may be conditioned to arrest the hydrolysis process as to maintain the properties and compositions present at the time the sample was taken from the oven or diffuser (e.g., the process vessel 110). For example, a sample may be refrigerated and frozen was stop the hydrolysis process until the offline assay measurements are performed. In at least one embodiment, the physical samples are taking from the hydrolysis process at or near in time when the online process spectral measurements of step 202 are collected. In at least one embodiment, the online process spectral measurements of step 202 may be collected concurrently with the physical samples of step 204. Data from the step 204 is added to the training data set.

According to at least one embodiment of the present disclosure, method 200 may include a step 206 of acquiring offline Raman spectra from a predetermined set of the physical samples before and/or after online spectra are acquired. In an embodiment, the step 206 may be performed when the physical samples collected online lack the variance needed for robust predictive process variable models. The physical samples selected should have a variance in at least one or more of the key process variables (e.g., analyte property and/or composition) for which Raman analysis will be used for real-time monitoring during production hydrolysis. The offline spectra may be from sample retains from various time points in previous hydrolysis batches, for example. Sample retains include physical samples taken from past hydrolysis batches and conditioned for storage. Such sample retains provide a record of past batches. Data from the step 206 may be added to the training data set. In at least one embodiment, the training data set may include solely Raman spectra data from the step 206, without any online spectral measurements of the step 202. In such an embodiment, the training data set and the resulting correlative model may be developed without using real-time, online Raman spectra data.

Alternatively, additionally or optionally, in an embodiment, the method 200 may include a step 208 of preparing (e.g., formulating) experimental samples and acquiring offline Raman spectra and offline assay measurements of the experimental samples. For example, the experimental samples may be created by adjusting the properties or compositions of the species within selected sample retain, conventionally referred to as “spiking.” Such spiking may include increasing or decreasing the presence or concentration of an analyte of interest. In at least one embodiment, adjusting the sample retains may follow a predetermined design of experiment (DoE) study in which selected process variables (e.g., analyte concentration) are purposefully and specifically set to predetermined values to statistically control the variance of the experimental samples and thereby determine the relative of effects of the variances, including correlated effects. Spiking may be used to expand the range of statistical models, to help mitigate Raman signal covariance between species in the fermenter, and to assist in modeling by amplifying Raman signal variances for correlation to individual offline assay values using a pre-determined amount of total acquisition time selected to generate a signal-to-noise ratio which is sufficient to produce the required measurement accuracy and precision. Data from the step 208 may be added to the training data set.

The method 200 further includes a step 210 of applying spectral preprocessing to the Raman spectral training data set to minimize non-correlative and covariant spectral variances of non-relevant species and properties (e.g., from species/analytes that are not of interest) across the Raman spectrum, while at the same time amplifying correlative changes due to the process variable(s) of importance (e.g., target analyte) for each modeled analyte. Spectral preprocessing may include mathematical manipulations of the training data set to increase spectral variance of a particular property or set of properties of the analyte of interest. In an embodiment, a series of optimizations are performed to discern the optimal preprocessing algorithm for a particular hydrolysis process. The spectral preprocessing may include a series of processes that can vary from data set to data set but may follow a series of rules and standard mathematical tools known to those skilled in the art of the present disclosure.

A step 212 of the method 200 includes determining whether to use a univariate and/or multivariate modeling methodology and applying the selected methodology to generate a correlative model that relates spectral changes (e.g., peaks (local maxima) and valleys (local minima)) in the preprocessed Raman spectral data acquisitions (e.g., spectral variances) from step 202 to changes observed in the values from the offline assay measurements from step 204 using conventional techniques. For example, the complexity of the hydrolysis process analyte constituent system, the quality of the Raman analyte peaks within the data, and/or the degree of spectral peak overlap may determine which modeling methodology is selected. Due to the inherent complexity of hydrolysis spectra, multivariate statistical modeling techniques may be more often applied relative to univariant techniques. The step 212 includes calculating a linear and/or non-linear correlation of the preprocessed Raman spectral data of the training data set to the values from the offline assay measurements for the analytes of interest to modeled. In at least one embodiment, the selected methodology may be applied to the offline Raman spectral data acquisitions of step 206 and/or step 208 in addition to the spectral data of step 202. The correlative model may include one or more analytes of interest depending on the scope of the Raman spectral data and the offline assay measurements. In an exemplary embodiment, a separate correlative model is generated for each analyte of interest. In such an embodiment, each separate model may be applied to the same preprocessed Raman spectral data. In a further embodiment, raw spectral Raman data may be acquired using different conditions that depend on a given target analyte and selected to optimize the quality of the spectral data relative to the target analyte.

When a statistically representative amount of Raman spectral data is captured and time matched to the offline assay measurement values, robust correlations are formed for which future predictions can be made when using the methods of the present disclosure to monitor the process. The robustness of correlative model is affected by the training data set and, for example, to the degree to which the training data set includes sufficiently variant spectra-assay combinations to capture the range of chemical, physical and compositional changes within the process from beginning to end of the hydrolysis process. This variance can be captured via natural and/or artificial means (e.g., using the step 208), as long as the samples represent the range of conditions within the process vessel, as described herein.

A step 214 of the method 200 includes iteratively refining the correlative model using modeling statistics to enhance correlation of the Raman spectra to the offline assay measurements values and/or reduce noise, bias and/or other unwanted artifacts in the correlative model. For example, the correlative model may be refined using statistical plots that show both the relative spectral residual and the concentration residual remaining from the Raman prediction. In addition, plots that display in (modeling) space vs. out of (modeling) space error of the predicted value may be utilized.

The step 214 may further include validating the correlative model by repeating at least steps 202 and 204 on one or more hydrolysis processes and assessing the whether the correlative model is adequately predictive. In such an embodiment, validation Raman spectra are acquired from the hydrolysis process and compared to the corresponding validating offline assay measurements. A separate training data set (e.g., a “validation” training data set) need not be prepared. In a further embodiment, the validation Raman spectra from the step 214 and the corresponding offline assay measurements may be added to the training data set and the remaining steps of the method 200 to further refine and improve the robustness of the correlative model. For example, when the validation Raman spectra include variances in the analytes of interest outside those present in the training data set, the training data set may be updated and the correlative model refined to include and model those variances, thereby improve the robustness of the correlative model.

The method 200 may further include a step 216 of applying the correlative model to subsequent online and/or offline data sets from future hydrolysis batches as to qualitatively and/or quantitatively predict analyte values (e.g., properties and/or concentrations) of interest from real-time, Raman spectral data collected from the future hydrolysis process in progress, for example, using the Raman system 100.

In at least one embodiment of the present disclosure, the measured, modeled and predicted analyte values of the method 200 may include at least one of degrees Brix (i.e., total dissolved sugar content by mass), pH, inulin concentration, furfural (e.g., hydroxymethyl furfural) concentration, sucrose concentration, glucose concentration, fructose and methanol concentrations, as non-limiting examples.

In the context of the present disclosure, a “prediction” is a conversion of Raman spectral data from online Raman analysis measurements using the correlative model to estimate or predict the true value of the property (or properties) of the analyte(s) of interest (e.g., chemical, physical and/or compositional). In an embodiment, predicted analyte values may be used to monitor the hydrolysis process in real time, without the need for offline measurements (e.g., by either Raman analysis or assay measurements) to alert an operator or a process control system that the analyte value has deviated from a desired range (e.g., based on threshold limits). In such an embodiment, the step 216 may include generating a signal, alarm or report to an operator of a process control system using the Raman system 100. The step 216 may further include performing an action to correct or compensate for the analyte value deviation or to abort the hydrolysis process based on the alert generated. One skilled in the art can identify other actions that may be performed based on the predicted analyte values, and such actions are within the scope of the present disclosure.

The method 200 of the present disclosure can provide real-time information about the hydrolysis process, enabling immediate adjustments to be made, for example, via a feedback loop step, to correct the properties and/or values of the constituents if any deviate from a desired or planned trajectory, which can lower costs and/or improve the quality of production. Additionally, the properties and/or values of the constituents will be known (at least as an aggregate) at the end of a particular batch such that additional offline measurements (e.g., quality control checks) need not be performed, further lowering costs.

During a hydrolysis process, using the methods of the present disclosure, the acquiring of Raman spectra should be set to a total acquisition time as to provide a certain level of precision with respect to a predicted or set of predicted chemical or physical measurement(s) following preprocessing and modeling, whether a univariate and/or a multivariate methodology is used. Each Raman spectra contains several spectral signatures from these chemical, physical and compositional properties to various signal-to-noise ratios, depending on the strength of the signal associated with a given property and on the noise within the spectrum at the relevant wavelength positions in the spectrum. Once spectral preprocessing is applied, as in step 210, the signals are then related to changes that occur in each property within the data set.

Chemometric modeling can often be influenced by the scale of the process being monitored. However, scale-independent multivariate models based on Raman spectral data obtained from bioreactor cultures of one or more different scales can be developed such that models based on Raman spectral data obtained at test scales (e.g., bench and/or pilot-scales) are accurate and precise at larger manufacturing-scale (e.g., 1000 L or greater) settings (see, e.g., U.S. Pat. No. 10,563,163). Likewise, accurate and precise, scale-independent, multivariate models based on Raman spectral data from hydrolysis processes of one or more different scales can be developed. Model development using smaller scale test reactors is generally faster and less costly, allowing more parameters to be tested and modeled often for a lower cost. By developing multivariate models based on Raman spectral data obtained across multiple different scales of hydrolysis processes, accurate determination and real-time optimization of key process parameters across a range of scales, including manufacturing-scales, is practical.

The systems and methods of the present disclosure have been applied to agave hydrolysis in a manufacturing-scale tequila production process. Consequently, Applicant has demonstrated that after pretreatments, key analyte parameters can be determined in real time during the in situ measurement of a hydrolysis process using embodiments of the Raman system 100 and the method 200 disclosed herein, including degrees Brix (i.e., total dissolved sugar content by mass), pH value, inulin concentration, sucrose concentration, glucose concentration, fructose concentration, and methanol concentration, as non-limiting examples. Nonetheless, one skilled in the art having the benefit of the present disclosure will recognize that other analytes are possible. Correlation of the correlative model was demonstrated using such known statistical metrics as a coefficient of determination (R2) and chemometric scores and loadings. In certain embodiments, other statistical metrics may be used including, but not limited to, root mean square error of validation (RMSEV), root mean square error of cross-validation (RMSECV), root mean square error of prediction (RMSEP), standard error of prediction (SEP), residual predictive deviation (RPD), and standard deviation (SD).

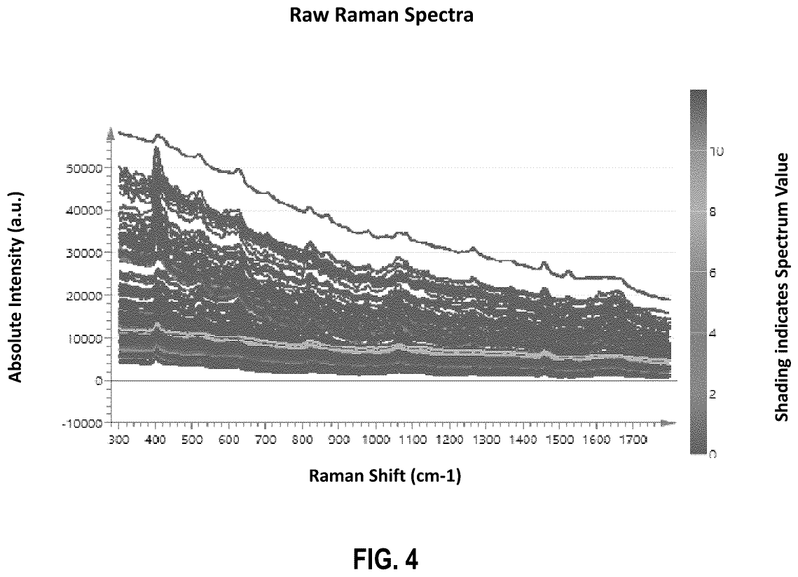

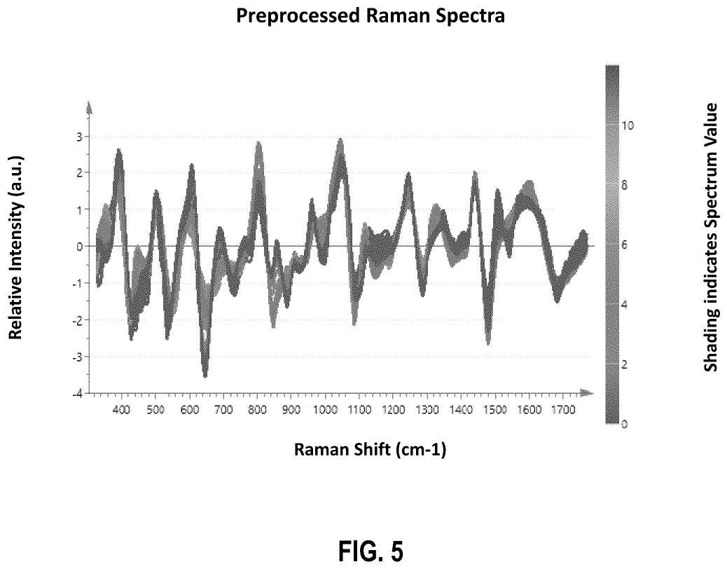

FIGS. 4 and 5 show examples of raw and preprocessed Raman spectra acquired throughout several batches of a hydrolysis process of raw agave stems using the Raman system 100. FIG. 4 presents raw Raman spectral data in which the gray-scale shading of each curve represents a given batch over time. FIG. 5 presents preprocessed Raman spectra of the spectral data of FIG. 4, in which the gray-scale shading of each curve represents a given batch over time. The raw spectral data were acquired over five (5) batches, one (1) spectrum every minute then summed for two (2) minutes for a total of three (3) spectra per sample for 182 total samples. The raw spectral data contains the raw spectral variance of both the captured analyte information within the batch (at each specific time point) and unwanted variation from other sources present in the process. To isolate and correlate the molecular signatures of interest, which include the desired compositional information of analytes of interest, spectral preprocessing was performed to remove unwanted variance and to amplify the spectral signatures of the analytes contained within the Raman spectrum as the batch evolved. The spectral changes (i.e., peaks and valleys) of the preprocessed spectra indicate changes in analyte concentrations.

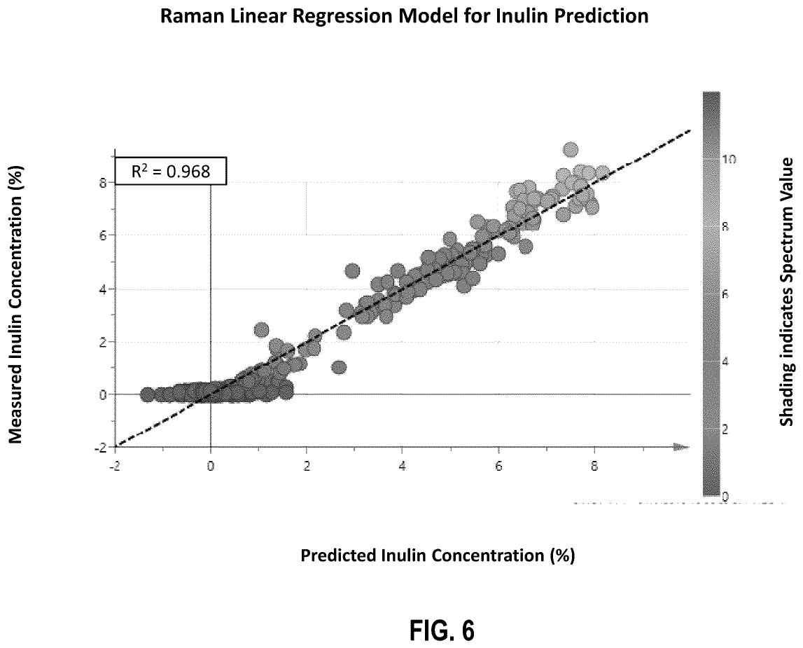

As an example, multivariate PLS analysis was performed on the preprocessed spectra of FIG. 5, which contain the multiple component spectral signatures, to extract each specific analyte and/or property of interest. In this example, the PLS algorithm was configured to statistically compare the changes from the known concentration values of inulin (e.g., acquired from the offline assays as described herein with respect to the method 200) with the online Raman spectral changes and, further, to determine how strong the correlation exists between both variances. The PLS algorithm was configured to deconstruct the changes into correlation metrics, which were plotted to identify relationships and to determination and/or assess the quality of the base model. FIGS. 6-9 show linear correlations between measured and predicted concentrations of predictive constituent models for inulin, fructose, glucose and sucrose, respectively.

FIG. 6 shows a linear regression of inulin concentration (in % v/v) between the measured offline assay values and predicted values using the validated correlative model. As shown in FIG. 6, the prediction precision across the range of inulin concentrations was excellent, having a coefficient of determination (R2) of 0.968. In the context of the present disclosure, a “prediction” is a conversion of Raman spectral data from online Raman analysis measurements using the correlative model to estimate or predict the true value of the property of the analyte of interest.

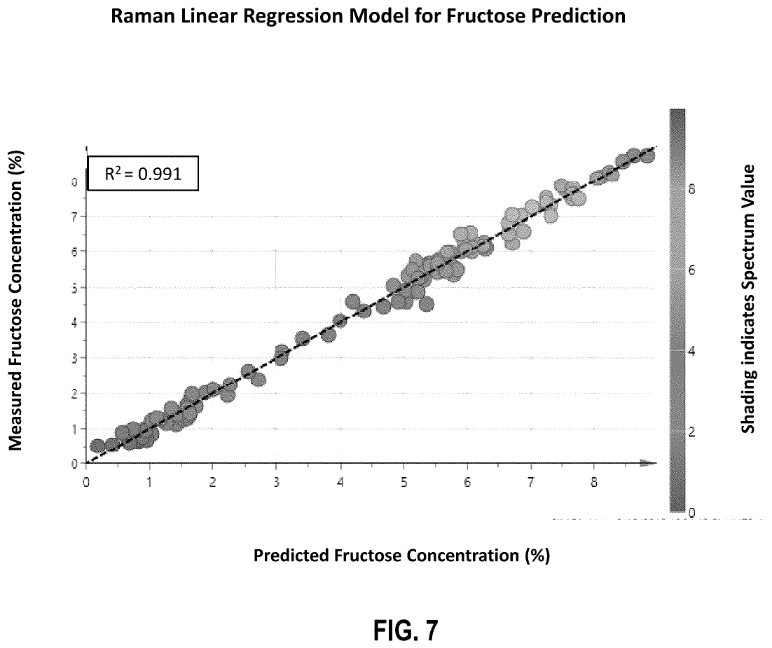

FIG. 7 shows a linear regression of fructose concentration (in % v/v) between the measured offline assay values and predicted values using the validated correlative model. As shown in FIG. 7, the prediction precision across the range of fructose concentrations was excellent, having a coefficient of determination (R2) of 0.991.

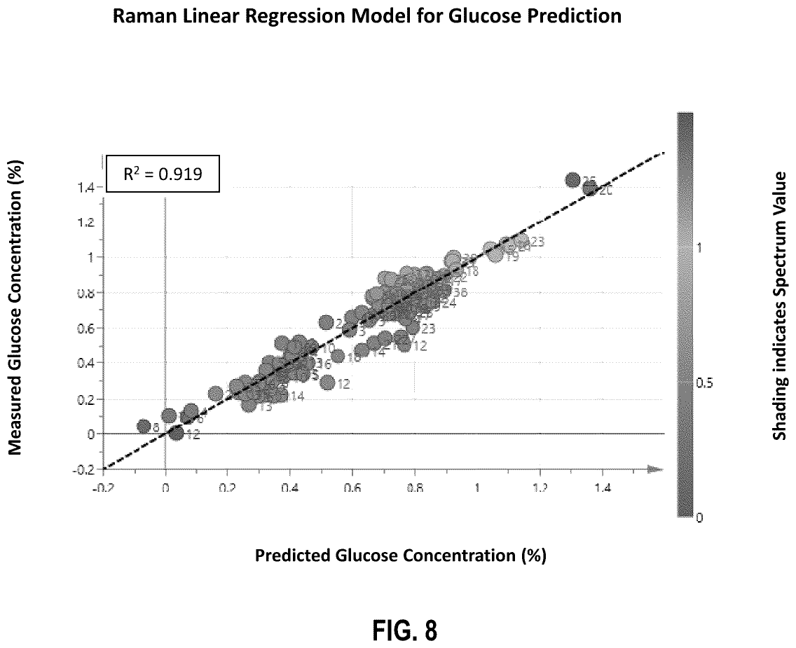

FIG. 8 shows a linear regression of glucose concentration (in % v/v) between the measured offline assay values and predicted values using the validated correlative model. As shown in FIG. 8, the prediction precision across the range of glucose concentrations was excellent, having a coefficient of determination (R2) of 0.919.

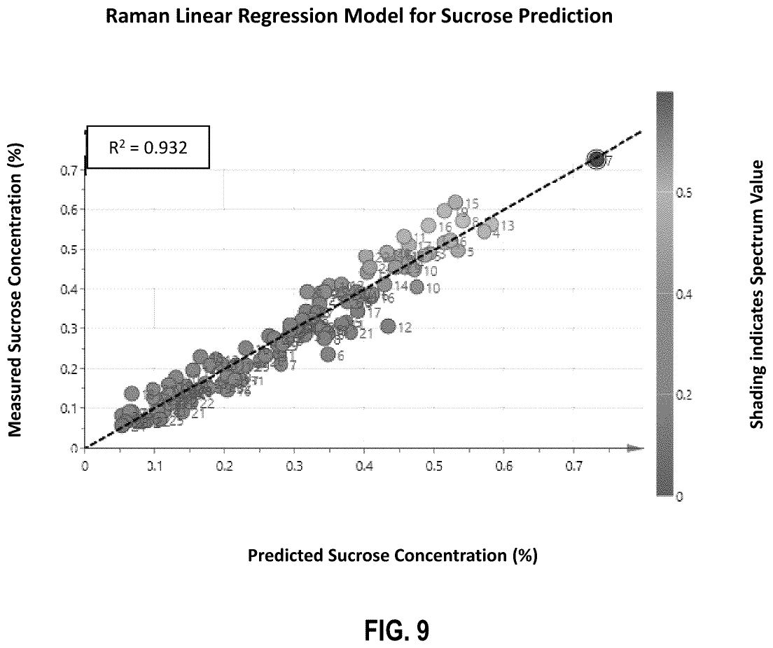

FIG. 9 shows a linear regression of sucrose concentration (in % v/v) between the measured offline assay values and predicted values using the validated correlative model. As shown in FIG. 9, the prediction precision across the range of sucrose concentrations was excellent, having a coefficient of determination (R2) of 0.932.

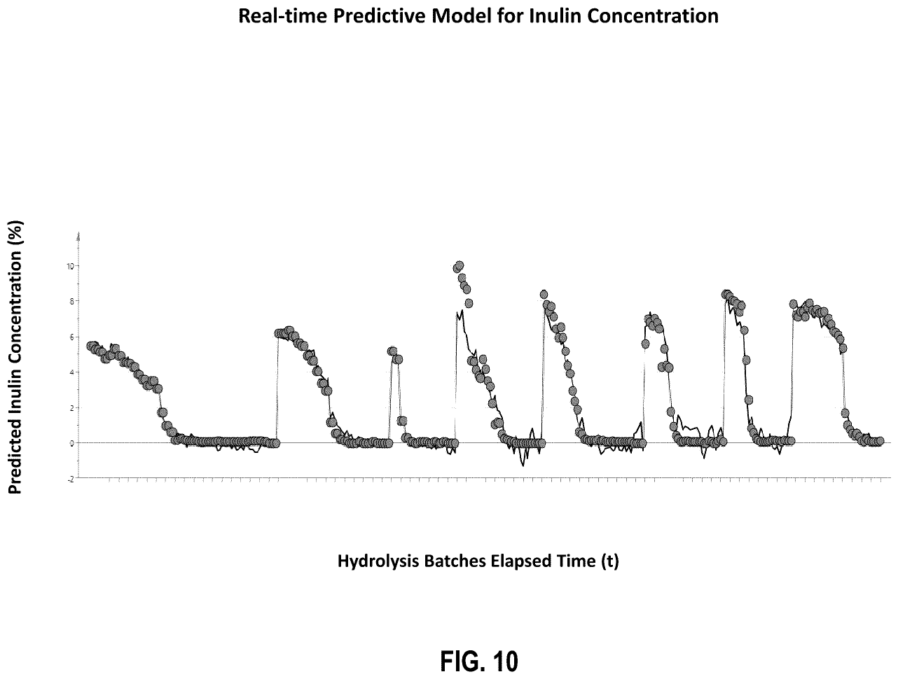

FIGS. 10-13 show correlations between measured and predicted concentrations of predictive constituent models over time for inulin, fructose, glucose and sucrose, respectively. For example, FIG. 10 shows a real-time prediction plot of inulin concentration (% v/v) during a series of eight (8) separate hydrolysis batches (t) using the validated correlative model of FIG. 6. As expected, within each batch, inulin concentration starts relatively high and then decreases as the inulin is hydrolyzed into simpler sugars. Again, as demonstrated in FIG. 10, the method 200 yields a correlative model demonstrating excellent correlation between measured offline assay samples, represented by circles, and real-time prediction values, represented by the solid line, of an inulin model according to the present disclosure.

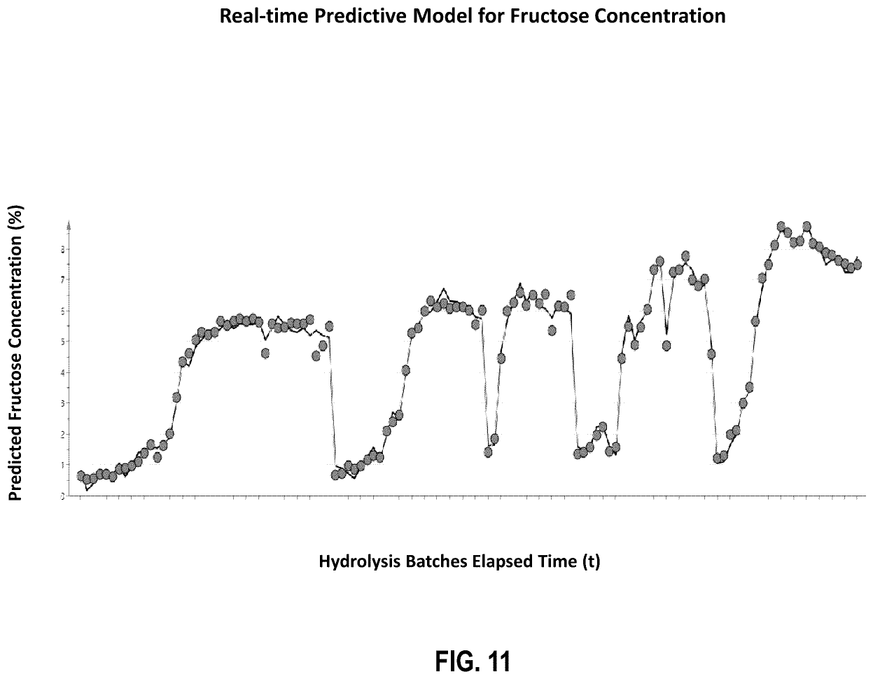

FIG. 11 shows a real-time prediction plot of fructose concentration (% v/v) during a series of five (5) separate hydrolysis batches (t) using the validated correlative model of FIG. 7. As expected, within each batch, fructose concentration starts relatively low and then increases as the inulin is hydrolyzed into simpler sugars. Again, as demonstrated in FIG. 11, the method 200 yields a correlative model demonstrating excellent correlation between measured offline assay samples, represented by circles, and real-time prediction values, represented by the solid line, of a fructose model according to the present disclosure.

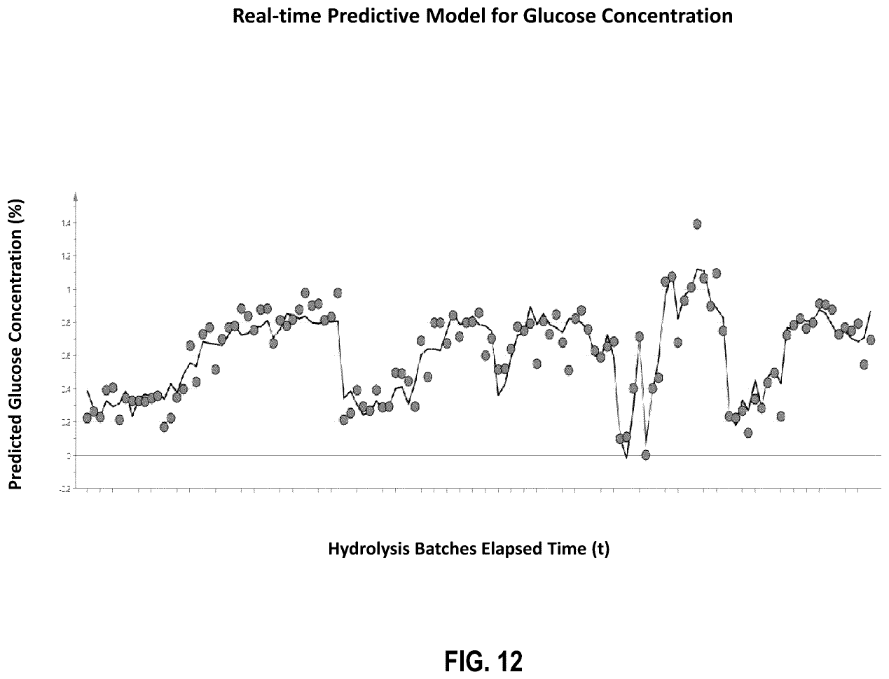

FIG. 12 shows a real-time prediction plot of glucose concentration (% v/v) during a series of five (4) separate hydrolysis batches (t) using the validated correlative model of FIG. 8. As expected, within each batch, glucose concentration starts relatively low and then increases as the inulin is hydrolyzed into simpler sugars. Again, as demonstrated in FIG. 12, the method 200 yields a correlative model demonstrating excellent correlation between measured offline assay samples, represented by circles, and real-time prediction values, represented by the solid line, of a glucose model according to the present disclosure.

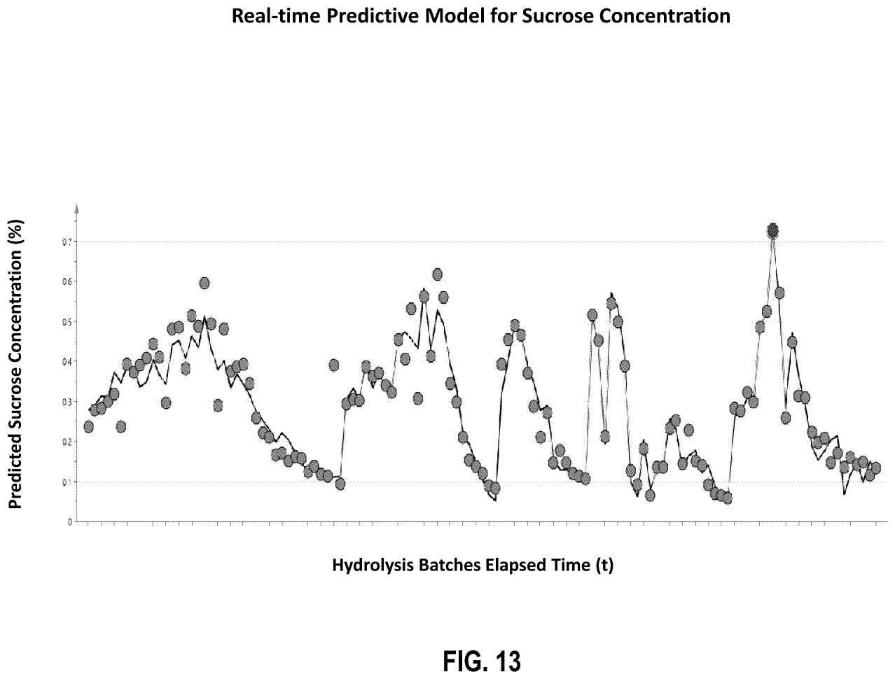

FIG. 13 shows a real-time prediction plot of sucrose concentration (% v/v) during a series of five (5) separate hydrolysis batches (t) using the validated correlative model of FIG. 9. As expected, within each batch, sucrose concentration starts relatively low, increases as the inulin is hydrolyzed into simpler sugars, and then decreases as the sucrose itself is hydrolyzed over time. Again, as demonstrated in FIG. 13, the method 200 yields a correlative model demonstrating excellent correlation between measured offline assay samples, represented by circles, and real-time prediction values, represented by the solid line, of a sucrose model according to the present disclosure.

At each stage, a correlative model may be developed using an embodiment of the method 200 to model stages of the tequila production process at a target stage by acquiring Raman spectra at that stage and correlating the acquired Raman spectra to offline assay measurements and/or to known standard samples (e.g., measured via another primary or secondary assay), as described herein in the context of the hydrolysis process. Consequently, at each stage, corrective actions may be taken based on the real-time, predictive values from online monitoring of each stage to improve quality, avoid lost batches and lower overall cost for the entire tequila making process.

### Fermentation Process Implementation

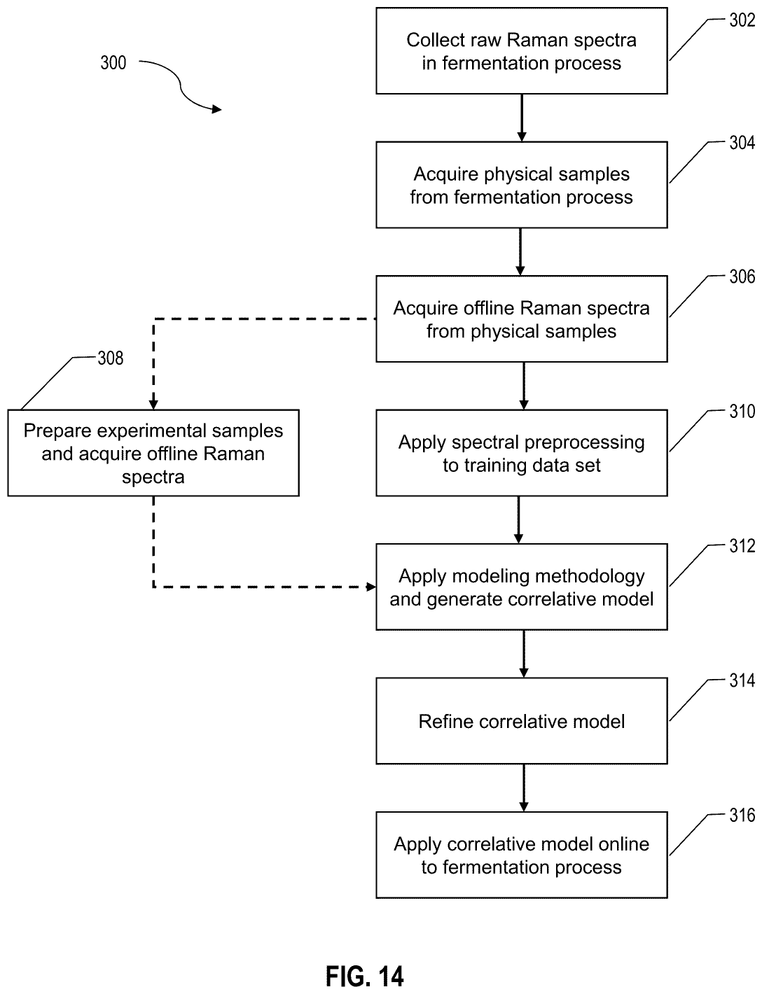

For example, the present disclosure includes a method 300 for using Raman analysis to model and determine the presence and concentration of multiple analytes within the agave fermentation process, as shown in FIG. 14. The method 300 may include a step 302 of generating a training data set by spectrally collecting a statistically large set of online or inline process spectral measurements of raw Raman spectra at different points in time while the fermentation process is in progress (e.g., in a fermentation vessel (or fermenter) 110 of FIG. 2) using the Raman system 100. The step 302 is analogous to the step 202 of the method 200; however, the particular analytes and process condition variation in the step 302 are selected for the specifics of fermentation of the hydrolyzed agave aguamiel, its constituents, and its by-products.

In at least one embodiment of the present disclosure, the measured, modeled and predicted analyte values of the method 300 may include at least one of degrees Brix (i.e., total dissolved sugar content by mass), pH, total acidity, concentrations of non-fermentable sugars (inulin, sucrose, other polysaccharides, etc.), fermentable sugars (e.g., glucose, fructose, etc.), organic acids (e.g., lactic acid, acetic acid, malic acid, etc.), volatile acidity (e.g., acetic acid, formic acid, butyric acid), non-volatile acids (e.g., gluconic acid, lactic acid concentration), polyols (e.g., glycerol, etc.), volatile alcohols (e.g., ethanol, methanol, 1-propanol), aldehydes and esters (e.g., furfural, acetaldehyde, ethyl acetate, etc.), and volatile fusel alcohols (e.g., isobutanol, 2-methyl-1-butanol, and 3-methyl-1-butanol, etc.), as non-limiting examples.

The method 300 may include a step 304 of acquiring physical samples during the fermentation process (e.g., from the fermenter 110) and performing offline and/or atline measurements of the analyte properties and/or compositions using an assay measurement device conventionally used to produce such values, including those prior art (e.g., non-Raman) devices and means described herein. As a non-limiting example related to the fermentation stage, physical samples may be acquired from the fermentation process batch every two hours over the course of multiple days as the process progresses (“typical” tequila fermentations last between 3 to 5 days, though they could last much longer). In at least one embodiment, the physical samples may be conditioned to arrest the fermentation process as to maintain the properties and compositions present at the time the sample was taken from the fermenter 110. For example, a sample may be taken from the fermenter and placed in a centrifuge or otherwise filtered to separate the active yeast from the other constituents, thereby stopping the fermentation process with the sample. As a further example, the sample may be refrigerated and frozen was stop the fermentation process until the offline assay measurements are performed. In at least one embodiment, the physical samples are taking from the fermentation process at or near in time when the online process spectral measurements of step 302 are collected. In at least one embodiment, the online process spectral measurements of step 302 may be collected concurrently with the physical samples of step 304. Data from the step 304 is added to the training data set.

According to at least one embodiment of the present disclosure, method 300 may include a step 306 of acquiring offline Raman spectra from a predetermined set of the physical samples before and/or after online spectra are acquired. In an embodiment, the step 306 may be performed when the physical samples collected online lack the variance needed for robust predictive process variable models. The physical samples selected should have a variance in at least one or more of the key process variables (e.g., analyte property and/or composition) for which Raman analysis will be used for real-time monitoring during production fermentation. The offline spectra may be from sample retains from various time points in previous fermentation batches, for example. Sample retains include physical samples taken from past fermentation batches and conditioned for storage. Such sample retains provide a record of past batches. Data from the step 306 may be added to the training data set. In at least one embodiment, the training data set may include solely Raman spectra data from the step 306, without any online spectral measurements of the step 302. In such an embodiment, the training data set and the resulting correlative model may be developed without using real-time, online Raman spectra data.

Alternatively, additionally or optionally, in an embodiment, the method 300 may include a step 308 of preparing (e.g., formulating) experimental samples specific to the fermentation process and acquiring offline Raman spectra and offline assay measurements of the experimental samples, in a manner analogous to the step 208 of the method 200. The method 300 further includes a step 310 of applying spectral preprocessing to the Raman spectral training data set to minimize non-correlative and covariant spectral variances of non-relevant species and properties (e.g., from species/analytes that are not of interest) across the Raman spectrum, while at the same time amplifying correlative changes due to the process variable(s) of importance (e.g., target analyte) for each modeled analyte, in a manner analogous to the step 210 of the method 200.

A step 312 of the method 300 includes determining whether to use a univariate and/or multivariate modeling methodology and applying the selected methodology to generate a correlative model that relates spectral changes (e.g., peaks (local maxima) and valleys (local minima)) in the preprocessed Raman spectral data acquisitions (e.g., spectral variances) from step 302 to changes observed in the values from the offline assay measurements from step 304 using conventional techniques, in a manner analogous to the step 212 of the method 200. A step 314 of the method 300 includes iteratively refining the correlative model using modeling statistics to enhance correlation of the Raman spectra to the offline assay measurements values and/or reduce noise, bias and/or other unwanted artifacts in the correlative model, in a manner analogous to the step 214 of the method 200. The method 300 enables the characterization, monitoring and controlling of the agave fermentation process as to produce tequila of a desired favor profile and quality.

### Distillation Process Implementation

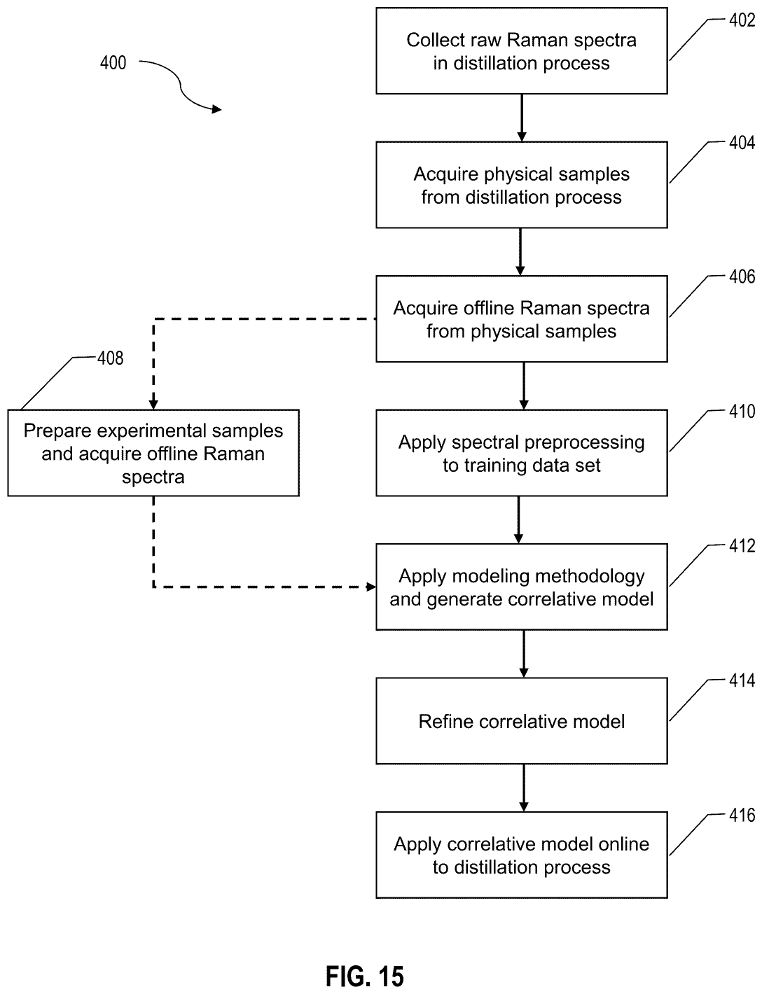

The present disclosure includes a method 400 for using Raman analysis to model and determine the presence and concentration of multiple analytes within the tequila distillation process, as shown in FIG. 15. The method 400 may include a step 402 of generating a training data set by spectrally collecting a statistically large set of online or inline process spectral measurements of raw Raman spectra at different points in time while the distillation process is in progress (e.g., in a distillation vessel 110 of FIG. 2, e.g., a distillation column) using the Raman system 100. The step 402 is analogous to the step 202 of the method 200; however, the particular analytes and process condition variation in the step 402 are selected for the specifics of distillation of the fermented agave must, its constituents, and its by-products.

In at least one embodiment of the present disclosure, the measured, modeled and predicted analyte values of the method 400 may include at least one of ethanol concentration, concentration of volatile compound (e.g., fusel alcohols, esters, aldehydes, acetate, methanol), pH, and concentration of non-volatile compounds (e.g., glycerol, etc.), as non-limiting examples.

The method 400 may include a step 404 of acquiring physical samples during the distillation process (e.g., from the vessel 110) and performing offline and/or atline measurements of the analyte properties and/or compositions using an assay measurement device conventionally used to produce such values, including those prior art (e.g., non-Raman) devices and means described herein. With respect to the distillation stage, physical samples may be acquired from the distillation process batch according to different process control regimens, depending on the type of still or distillation column used, the scale of production, and desired product characteristics, as non-liming examples. In at least one embodiment, the physical samples are taking from the distillation process at or near in time when the online process spectral measurements of step 402 are collected. In at least one embodiment, the online process spectral measurements of step 402 may be collected concurrently with the physical samples of step 404. Data from the step 404 is added to the training data set.

According to at least one embodiment of the present disclosure, method 400 may include a step 406 of acquiring offline Raman spectra from a predetermined set of the physical samples before and/or after online spectra are acquired in a manner analogous to the step 210 of the method 200, as described herein. In an embodiment, the step 306 may be performed when the physical samples collected online lack the variance needed for robust predictive process variable models. The physical samples selected should have a variance in at least one or more of the key process variables (e.g., analyte property and/or composition) for which Raman analysis will be used for real-time monitoring during production distillation. The offline spectra may be from sample retains from various time points in previous distillation batches, for example. Sample retains include physical samples taken from past distillation batches and conditioned for storage. Such sample retains provide a record of past batches. Data from the step 406 may be added to the training data set. In at least one embodiment, the training data set may include solely Raman spectra data from the step 406, without any online spectral measurements of the step 402. In such an embodiment, the training data set and the resulting correlative model may be developed without using real-time, online Raman spectra data.

Alternatively, additionally or optionally, in an embodiment, the method 400 may include a step 408 of preparing (e.g., formulating) experimental samples specific to the distillation process and acquiring offline Raman spectra and offline assay measurements of the experimental samples, in a manner analogous to the step 208 of the method 200. The method 400 further includes a step 410 of applying spectral preprocessing to the Raman spectral training data set to minimize non-correlative and covariant spectral variances of non-relevant species and properties (e.g., from species/analytes that are not of interest) across the Raman spectrum, while at the same time amplifying correlative changes due to the process variable(s) of importance (e.g., target analyte) for each modeled analyte, in a manner analogous to the step 210 of the method 200.

A step 412 of the method 400 includes determining whether to use a univariate and/or multivariate modeling methodology and applying the selected methodology to generate a correlative model that relates spectral changes (e.g., peaks (local maxima) and valleys (local minima)) in the preprocessed Raman spectral data acquisitions (e.g., spectral variances) from step 402 to changes observed in the values from the offline assay measurements from step 404 using conventional techniques, in a manner analogous to the step 212 of the method 200. A step 414 of the method 400 includes iteratively refining the correlative model using modeling statistics to enhance correlation of the Raman spectra to the offline assay measurements values and/or reduce noise, bias and/or other unwanted artifacts in the correlative model, in a manner analogous to the step 214 of the method 200. The method 400 enables the characterization, monitoring and controlling of the agave distillation process as to produce tequila of a desired favor profile and quality.

One skilled in the art of Raman spectroscopic analysis, having the benefit of the present disclosure, will recognize that using the Raman system 100 the method 200 may be adapted and applied to each stage of the tequila production process from extraction to filtering. While various embodiments of a Raman spectroscopic analysis system and methods generating chemometric models for using the same have been described in considerable detail herein, the embodiments are merely offered by way of non-limiting examples of the disclosure described herein. It will therefore be understood that various changes and modifications may be made, and equivalents may be substituted for elements thereof, without departing from the scope of the disclosure. The present disclosure is not intended to be exhaustive or to limit the scope of the subject matter of the disclosure.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. Other sequences of steps may be possible and thus remain within the scope of the present disclosure.

Claimed is:

1. A method of characterizing and monitoring an agave fermentation process, the method comprising:
acquiring training Raman spectra of an agave juice solution within a vessel at different times during the agave fermentation process to generate a training data set;
applying spectral preprocessing to the training data set such that non-correlative and covariant changes due to non-relevant species and/or properties are minimized and correlative changes due to a target analyte are amplified;
acquiring physical samples from the agave fermentation process near in time to the acquired training Raman spectra;
performing offline measurements on the physical samples of at least one property and/or composition of the target analyte using an assay measurement technique;
generating a correlative model of the target analyte such that spectral changes in the training data set correlate with the offline measurements of the at least one property and/or composition of the target analyte;
acquiring online Raman spectra of a subsequent batch run of the agave fermentation process within the vessel at different times during the batch run to generate a process data set; and
applying the correlative model to the process data set to qualitatively and/or quantitatively predict a value of the at least one property and/or composition of the target analyte.

2. The method of claim 1, wherein the target analyte includes more than one species or property of the agave fermentation process.

3. The method of claim 1, wherein the acquiring of the training Raman spectra is performed inline, in real time, during operation of the agave fermentation process.

4. The method of claim 3, the method further comprising acquiring offline Raman spectra from a predetermined set of the physical samples before and/or after online Raman spectra are acquired.

5. The method of claim 4, wherein data from the acquired offline Raman spectra are included in the training data set.

6. The method of claim 1, wherein the physical samples are acquired concurrently with the training Raman spectra.

7. The method of claim 1, the method further comprising preparing experimental samples and acquiring offline Raman spectra and offline assay measurements of the experimental samples, wherein data of the experimental samples are included in the training data set.

8. The method of claim 7, wherein the experimental samples are prepared according to a predetermined design of experiment in which selected analyte variables are prepared to predetermined values such that variance of the experimental samples is to statistically controlled.

9. The method of claim 1, wherein the spectral preprocessing includes a series of optimizations adapted to discern an optimal preprocessing algorithm for the agave fermentation process.

10. The method of claim 1, wherein the corelative model is generated using a univariate modeling methodology.

11. The method of claim 1, wherein the corelative model is generated using a multivariate modeling methodology.

12. The method of claim 1, the method further comprising iteratively refining the correlative model using modeling statistics such that correlation of the training data set to the offline measurements is increased.

13. The method of claim 1, the method further comprising generating a signal, alarm or report when the value of the at least one property and/or composition of the target analyte deviates from a desired range based on a threshold limit.

14. The method of claim 1, wherein the target analyte includes at least one of degrees Brix, pH, inulin, fructose, glucose, sucrose, total acidity, acetic acid, gluconic acid, lactic acid, malic acid, formic acid, butyric acid, glycerol, ethanol, methanol, 1-propanol, furfural, acetaldehyde, ethyl acetate, isobutanol, 2-methyl-1-butanol, and 3-methyl-1-butanol.

15. A computer program product comprising a non-transitory machine-readable storage medium encoding instructions that, when executed by one or more programmable processors, cause the one or more programmable processors to perform operations comprising:
acquiring online Raman spectra of an agave fermentation process within a vessel at different times during the fermentation process to generate a training data set;
applying spectral preprocessing to the training data set such that non-correlative and covariant changes due to non-relevant species and/or properties are minimized and correlative changes due to a target analyte are amplified;
generating a corelative model of the target analyte such that spectral changes in the training data set associated with at least one property and/or composition of the target analyte corelate with offline assay measurements of physical samples taken from the fermentation process near in time to the acquired Raman spectra;
acquiring a subsequent online Raman spectrum during a subsequent batch run of the fermentation process within the vessel; and
applying the correlative model to the subsequent online Raman spectra to qualitatively and/or quantitatively predict a value of the least one property and/or composition of the target analyte.

16. The computer program product of claim 15, wherein the operations further comprise generating a signal, alarm or report when the value of the at least one property and/or composition of the target analyte deviates from a desired range based on a threshold limit.

17. The computer program product of claim 15, wherein the target analyte includes at least one of degrees Brix, pH, inulin, fructose, glucose, sucrose, total acidity, acetic acid, gluconic acid, lactic acid, malic acid, formic acid, butyric acid, glycerol, ethanol, methanol, 1-propanol, furfural, acetaldehyde, ethyl acetate, isobutanol, 2-methyl-1-butanol, and 3-methyl-1-butanol.

18. A Raman analysis system, the system comprising:
a light source configured to emit excitation light;
an optical probe coupled to the light source via an optical cable such that the excitation light is emitted from a probe head of the probe into a sample volume, wherein the probe is configured to receive and transmit a Raman signal from the sample volume via the optical cable or another optical cable;
a spectrograph configured to spectrally separate the transmitted Raman signal;
a detector configured to receive the separated Raman signal and convert the separated Raman signal into a Raman spectrum; and
a controller configured to:
acquire online Raman spectra of an agave fermentation process within a vessel at different times during the pressing process by actuating the light source to emit the excitation light and receiving the Raman spectrum from the detector;
apply a correlative model to the Raman spectra to qualitatively and/or quantitatively predict a value of a property and/or composition of a target analyte, wherein the correlative model is adapted such that spectral changes associated with properties and/or compositions of the target analyte in previously acquired Raman spectra correlate with offline assay measurements of physical samples taken from a previous batch run of the fermentation process; and
monitor the fermentation process by periodically acquiring subsequent online Raman spectra.

19. The system of claim 18, wherein the controller is further configured to generate a signal, alarm or report when the value of the at least one property and/or composition of the target analyte deviates from a desired range based on a threshold limit.

20. The method of claim 18, wherein the target analyte includes at least one of degrees Brix, pH, inulin, fructose, glucose, sucrose, total acidity, acetic acid, gluconic acid, lactic acid, malic acid, formic acid, butyric acid, glycerol, ethanol, methanol, 1-propanol, furfural, acetaldehyde, ethyl acetate, isobutanol, 2-methyl-1-butanol, and 3-methyl-1-butanol.
